(19) United States
(12) Patent Application Publication
(10) Pub. No.: US 2005/0187441 A1
(43) Pub. Date: Aug. 25, 2005

(54) Laser-scanning examination apparatus

(76) Inventors: Kenji Kawasaki, Hachioji-shi (JP); Hiroshi Sasaki, Nerima-ku (JP); Kenichi Koyama, Sagamihara-shi (JP); Atsuhiro Tsuchiya, Hachioji-shi (JP); Yoshihiro Kawano, Hachioji-shi (JP)

Correspondence Address: KENYON & KENYON, 1500 K STREET NW, SUITE 700, WASHINGTON, DC, 20005, US

(21) Appl. No.: 11/035,075

(22) Filed: Jan. 14, 2005

(30) Foreign Application Priority Data
Jan. 19, 2004 (JP) 2004-011142
Apr. 7, 2004 (JP) 2004-113017

(51) Int. Cl. A61B 5/00; G03C 5/16

(52) U.S. Cl. 600/315; 250/580

(57) ABSTRACT

A laser-scanning examination apparatus includes a laser light source; an optical fiber through which laser light generated in the laser light source is transmitted; a scan head including a casing for housing a laser scanning unit that scans the laser light transmitted by the optical fiber on a specimen, an objective unit for imaging the laser light scanned by the laser scanning unit onto the specimen; an optical detector for detecting returning light that returns from the specimen to the interior of the casing via the objective unit; a stage for mounting the specimen; and an arm that supports the scan head so that the position and orientation of the objective unit with respect to the stage can be adjusted. The optical detector is provided on an outer surface of the casing except for the surface located in the opposite direction from the direction in which the arm extends from the casing. The laser-scanning examination apparatus has a compact head unit and allows improved ease-of-use by securing a large space around the head unit.

### BACKGROUND OF THE INVENTION

1. Field of the Invention

The present invention relates to a laser-scanning examination apparatus.

2. Description of Related Art

As one example of this kind of laser-scanning examination apparatus in the related art, the laser microscope disclosed in Japanese Unexamined Patent Application Publication No. 2000-330029 (paragraph [0016] etc.) is known.

This laser microscope has a configuration in which, using coherent light, light from a specimen is detected by a detector via an optical path splitting member interposed between a scanning optical system and an objective lens. This arrangement is advantageous in that it is possible to keep the number of reflections and transmissions to a minimum when guiding the light from the specimen to the detector, thus keeping the optical losses to a minimum.

The laser microscope disclosed in Japanese Unexamined Patent Application Publication No. 2004-330029 is a comparatively large microscope. In order to carry out in-vivo examination of specimens such as small experimental animals, it is necessary to carry out various positional and orientational alignments of the objective lens of the examination apparatus with respect to the specimen. Furthermore, it is essential to minimize the size of a head part in the vicinity of the specimen.

### BRIEF SUMMARY OF THE INVENTION

It is a first object of the present invention to provide a laser-scanning examination apparatus that can secure the head part in a suitable position or orientation, depending on the type of specimen. It is a second object of the present invention to provide a laser-scanning examination apparatus that can ensure increased space around the head part to improve the operability, while keeping the size of the head part to a minimum.

To realize the above-described objects, the present invention provides the following features.

The present invention provides a laser-scanning examination apparatus including a laser light source; an irradiation optical fiber through which laser light generated in the laser light source is transmitted; a scan head including a laser scanning unit for scanning the laser light transmitted through the irradiation optical fiber on a specimen, a casing for housing the laser scanning unit, and an objective unit for imaging the laser light scanned by the laser scanning unit onto the specimen; an optical detector for detecting returning light that returns from the specimen to the interior of the casing via the objective unit; a stage for mounting the specimen; a base on which the stage is provided; a support stand having a longitudinal axis extending from the base; an arm that extends from the support stand in a direction orthogonal to the longitudinal axis thereof; and a moving mechanism disposed between the arm and the scan head. The moving mechanism moves the scan head relative to the arm.

The scan head may be attached to the moving mechanism at an outer surface of the casing orthogonal to an optical axis of the objective unit.

The laser-scanning examination apparatus may also include a support stand tilting mechanism for tilting the support stand about a horizontal axis thereof.

The moving mechanism may include an X-axis and Y-axis moving mechanism for moving the scan head relative to the arm in two directions orthogonal to the optical axis of the objective unit.

The moving mechanism may include a tilting mechanism for adjusting the tilt angle of the scan head with respect to the arm.

The laser-scanning examination apparatus may also include a focus adjusting mechanism for relatively moving the objective unit along the optical axis thereof with respect to the casing.

The optical detector may be provided on an outer surface of the casing except for the surface positioned in a direction opposite to the direction in which the arm extends from the casing.

Preferably, the optical detector is secured to the casing in a direction extending along the arm.

The invention may also include a slider that is moveable upwards and downwards along the support stand. In this case, the base is disposed horizontally, the support stand extends vertically from the base, the arm is attached to the slider, and the optical detector is attached to the outer surface of the casing so as to extend in the direction of the support stand.

Preferably, the casing is provided with an inclined surface formed so as to taper towards the objective unit.

The optical detector may be disposed at the opposite side of the objective unit from the stage.

The optical detector may be positioned higher than the top surface of the casing.

The laser-scanning examination apparatus may also include a connecting part for connecting to the optical detector, the detection part being provided in an outer surface of the casing except for the surface located in the opposite direction from the direction in which the arm extends from the casing.

The laser-scanning microscope apparatus may also include a light-path splitting unit, in the connecting part, for splitting the light path. In this case, the optical detector includes a plurality of detectors for detecting light in the respective split light paths.

The connecting part may be disposed towards the support stand side of the optical axis of the objective unit.

The laser-scanning examination apparatus according to the invention may also include a detection optical fiber that connects the connecting part and the optical detector.

The laser-scanning examination apparatus according to the invention preferably also includes a splitting unit for splitting off returning light from the optical path between the objective unit and the laser scanning unit and for directing the split-off light towards the optical detector.

The laser-scanning examination apparatus according to the invention may also include a splitting unit for splitting off returning light from the optical path between the objective unit and the laser scanning unit and for directing the split-off light towards the optical detector; and a detection optical fiber that connects the connecting part and the optical detector. In this case, the connecting part is disposed near the position where the irradiation optical fiber is connected to the casing, and part of the detection optical fiber is disposed inside the casing so that one end thereof opposes the splitting unit.

According to the present invention, by reducing the size of the scan head, the ease-of-use is enhanced and the ability to replace the specimen, such as a relatively small experimental animal, is improved. In addition, by disposing a relatively large optical detector so that it does not obstruct the working space of the operator, a laser-scanning examination apparatus that facilitates examination can be provided.

### BRIEF DESCRIPTION OF THE SEVERAL VIEWS OF THE DRAWINGS

FIG. 1 is a plan view of a laser-scanning examination apparatus according to a first embodiment of the present invention.

FIG. 2 is a front elevational view of the laser-scanning examination apparatus in FIG. 1.

FIG. 3 is a side elevational view of the laser-scanning examination apparatus in FIG. 1.

FIG. 4 is a side elevational view of a modification of the laser-scanning examination apparatus in FIG. 1, in which two optical detectors are provided.

FIG. 5 is a side elevational view, like the modification shown in FIG. 4, in which a scanning head and an optical detector are connected by an optical fiber.

FIG. 6 is a plan view showing another modification of the laser-scanning examination apparatus in FIG. 1.

FIG. 7 is a front elevational view of the laser-scanning examination apparatus in FIG. 6.

FIG. 8 is a side elevational view of the laser-scanning examination apparatus in FIG. 6.

FIG. 9 is a front elevational view of another modification of the laser-scanning examination apparatus in FIG. 1.

FIG. 10 is a front elevational view of another modification like that in FIG. 9.

FIG. 11 is a side elevational view of another modification like that in FIG. 9.

FIG. 12 is a side elevational view of another modification like that in FIG. 9.

FIG. 13 is a side elevational view of another modification like that in FIG. 9.

FIG. 14 is a side elevational view showing a modification of the position at which the detector is installed.

FIG. 15 is a side elevational view showing another modification like that in FIG. 14.

FIG. 16 is a side elevational view showing another modification like that in FIG. 14.

FIG. 17 is a side elevational view showing another modification in which the optical detector is positioned above the casing.

FIG. 18 is a schematic diagram of a laser-scanning examination apparatus according to a second embodiment of the present invention.

FIG. 19 is a drawing for explaining the casing moving mechanism of the laser-scanning examination apparatus in FIG. 18.

FIG. 20 shows a modification of the casing moving mechanism in FIG. 19.

FIG. 21 is a schematic diagram of a laser-scanning examination apparatus according to a third embodiment of the present invention.

### DETAILED DESCRIPTION OF THE INVENTION

### First Embodiment

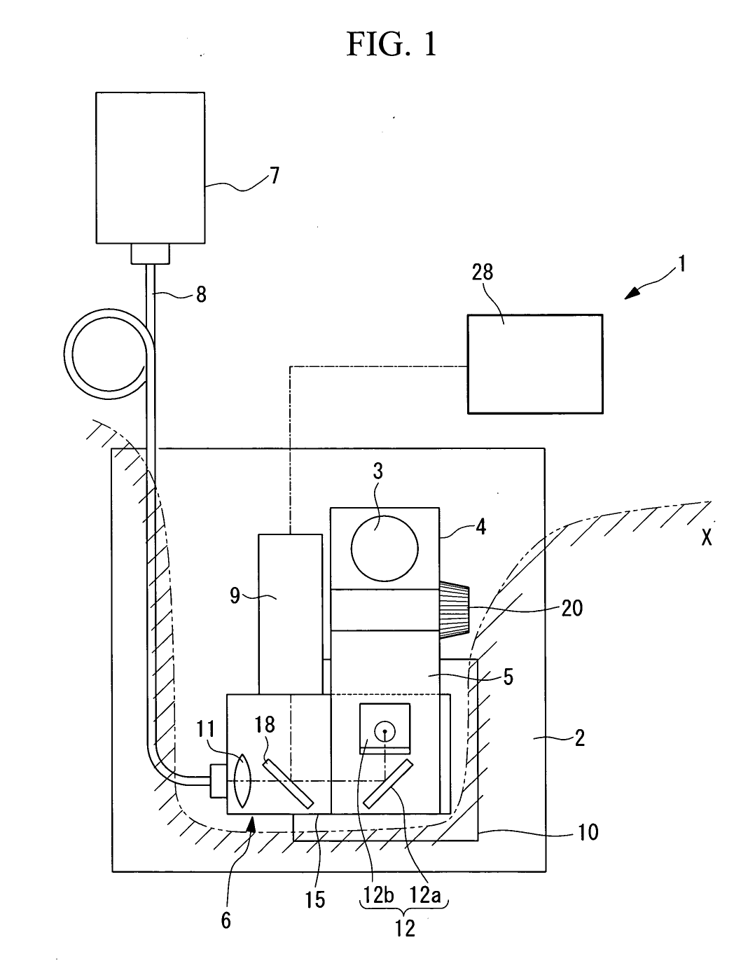

A laser-scanning examination apparatus according to a first embodiment of the present invention will be described below with reference to FIGS. 1 to 3.

As shown in FIG. 1, a laser-scanning examination apparatus according to this embodiment includes a base 2 that is positioned horizontally, a support stand 3 that extends vertically upwards from the upper surface of the base 2, a slider 4 that is positioned on the support stand 3 so as to be moveable upward and downward, an arm 5 that extends horizontally from the slider 4, a scan head 6 that is secured to the end of the arm 5, a laser light source 7 disposed externally, an optical fiber 8 that connects the laser light source 7 and the scan head 6, and an optical detector 9 that is secured to the scan head 6.

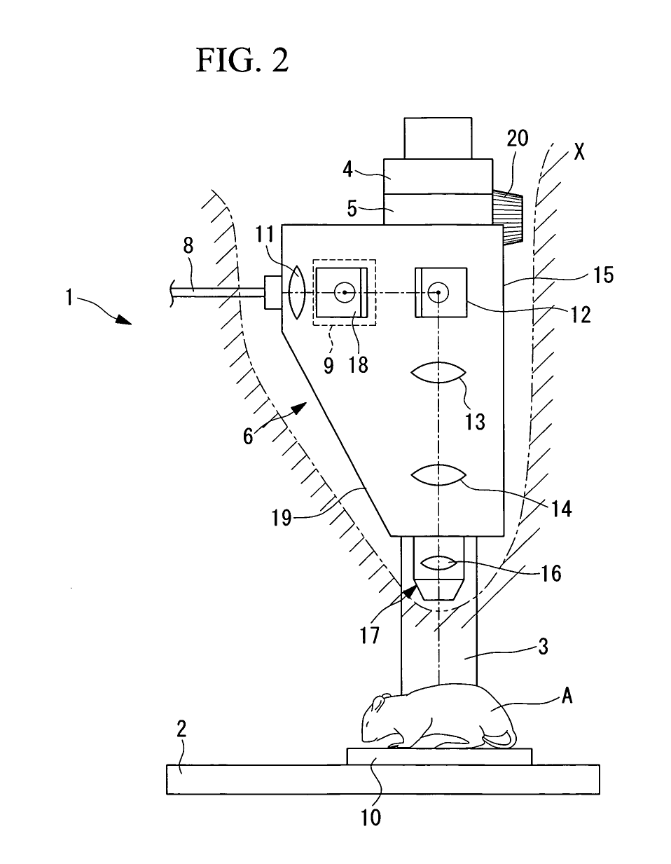

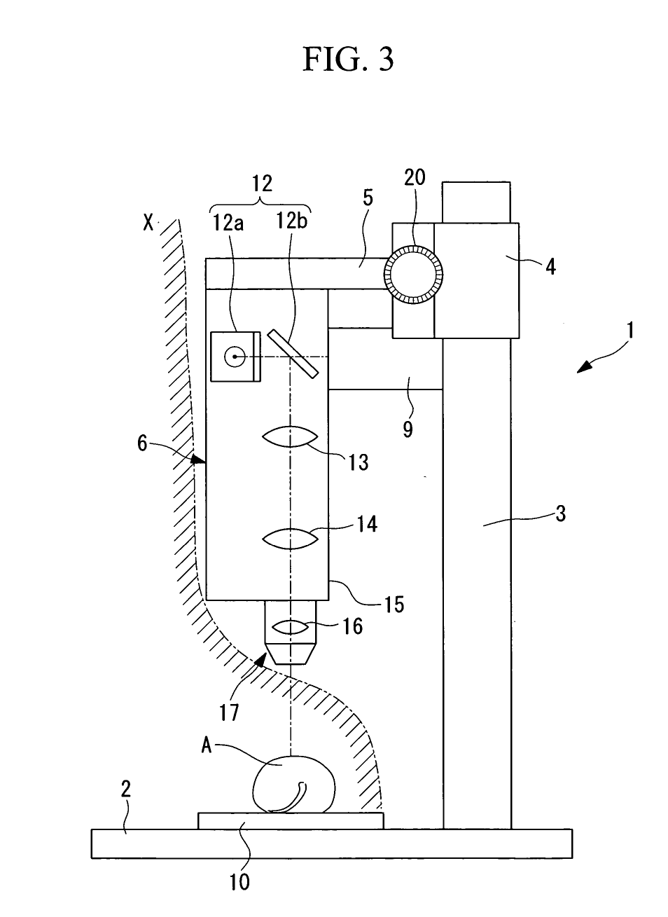

As shown in FIG. 2 and FIG. 3, a stage 10 for mounting a specimen A, such as a small experimental animal like a rat or a mouse, is provided on the base 2. The stage 10 can move the specimen A upwards and downwards, as well as horizontally, and is also configured so as to be rotatable around a vertical axis.

The scan head 6 includes, inside a casing 15 thereof, a collimator lens 11 for converting the laser light conveyed by the optical fiber 8 into collimated light, a laser scanning unit 12 that deflects the laser light in two horizontal directions by a pair of galvano mirrors 12a and 12b that rotate around two orthogonal axes, a pupil projection lens 13 that forms an intermediate image by focusing the laser light emitted from the laser scanning unit 12, and an imaging lens 14 that re-collimates the laser light forming the intermediate image by the pupil projection lens 13.

An objective unit 17, which includes an objective lens 16 for re-imaging the laser light emitted from the imaging lens 14 onto the specimen A, is detachably fitted to the lower end of the casing 15 of the scan head 6.

Also, the optical detector 9 is secured to the side face at the support stand side of the casing 15 of the scan head 6. The optical detector 9 is, for example, a photomultiplier tube and has comparatively large dimensions compared to the scan head 6. A dichroic mirror 18 for splitting off from the optical path the fluorescence returning from the specimen A via the objective lens 16, the imaging lens 14, the pupil projections lens 13, and the laser scanning unit 12 is provided inside the casing 15. The optical detector 9 is designed to detect the fluorescence split off from the optical path by the dichroic mirror 18. Reference numeral 28 in the drawing represents a monitor for displaying images captured by the optical detector 9.

Furthermore, the casing 15 of the scan head 6 is provided with an inclined surface 19 at the lower part thereof. The inclined surface 19 forms a taper on the casing 15 towards the lower end where the objective unit 17 is provided. With this arrangement, a working space X in the vicinity of the stage 10 can be increased, which makes it possible to facilitate operations such as manipulating the specimen A and so forth.

A focusing knob 20 is provided on the slider 4. The operator turns this knob 20 to move the arm 5 and the scan head 6 secured to the end of the arm 5 upwards and downwards with respect to the base 2, which allows the objective unit 17 to be moved closer to or further away from the examination site of interest in the specimen A mounted on the stage 10.

The arm 5 is configured so that the slider 4, the arm 5, and the scan head 6 rotate about the axis of the support stand 3.

Moreover, the operator normally operates the apparatus at the opposite side of the scan head 6 from the support stand 3 (hereinafter referred to as the front side). FIG. 2 shows a view from the front side of the laser-scanning examination apparatus 1 according to this embodiment, and FIG. 3 shows a view from the right side thereof.

A description follows of the operation of the laser-scanning examination apparatus 1 according to this embodiment, having such a configuration.

With the laser-scanning examination apparatus 1 according to this embodiment, laser light from the laser light source 7 is conveyed by the optical fiber 8, enters the casing 15 of the scan head 6, and passes through the collimator lens 11, the laser scanning unit 12, the pupil projection lens 13, the imaging lens 14, and the objective lens 16 to illuminate the specimen A. Since the operation of the laser scanning unit 12 causes the illumination position of the laser light on the specimen A to be scanned, the laser light can illuminate a predetermined region of the examination site of the specimen A. Fluorescence is then produced by the specimen A illuminated by the laser light, and the fluorescence produced returns via the objective lens 16, the imaging lens 14, the pupil projection lens 13, and the laser scanning unit 12 and is detected by the optical detector 9 when split off from the optical path by the dichroic mirror 18.

By illuminating various positions of the specimen A with the laser light using the laser scanning unit 12 and detecting the fluorescence returning from each position, a fluorescence image of a predetermined region of the examination site of the specimen A can be obtained, and this image can be examined on the monitor 28.

In this examination operation, the operator sets the specimen A on the stage 10 and operates a rotation mechanism (not shown in the drawings) provided between the support stand 3 and the slider 4 to position the scan head 6 approximately at the position of the specimen A. Also, by turning the focus knob 20 to move the slider 4 along the support stand 3, the examination site can be brought into focus. Generally, these operations are carried out in the working space X at the opposite side of the scan head 6 from the support stand 3.

With the laser-scanning examination apparatus 1 according to this embodiment, since the optical detector 9, which is of comparatively large dimensions, is fixed to the side surface of the casing 15 of the scan head 6 at the support stand 3 side (in other words, the rear surface), it does not extend into the working space X. Thus, when the operator carries out the examination operation described above, it is possible to prevent the working space X from being restricted by the optical detector 9. As a result, a relatively large working space X is secured around the scan head 6 and the stage 10, which facilitates the examination carried out by the operator. This applies not only in the case where the operator works at the front side but also in the cases where the operator works at the left side or the right side.

With the laser-scanning examination apparatus 1 according the embodiment shown in FIGS. 1 to 3, the optical fiber 8 joining the laser light source 7 and the casing 15 of the scan head 6 is connected at the left side when viewed from the front of the scan head 6; however, since the shape of the optical fiber 8 can be changed relatively freely, it can be arranged in any shape that does not hinder the operation. Also, the connection location of the optical fiber 8 from the laser light source 7 to the casing 15 of the scan head 6 can also be arranged at the surface at the support stand 3 side.

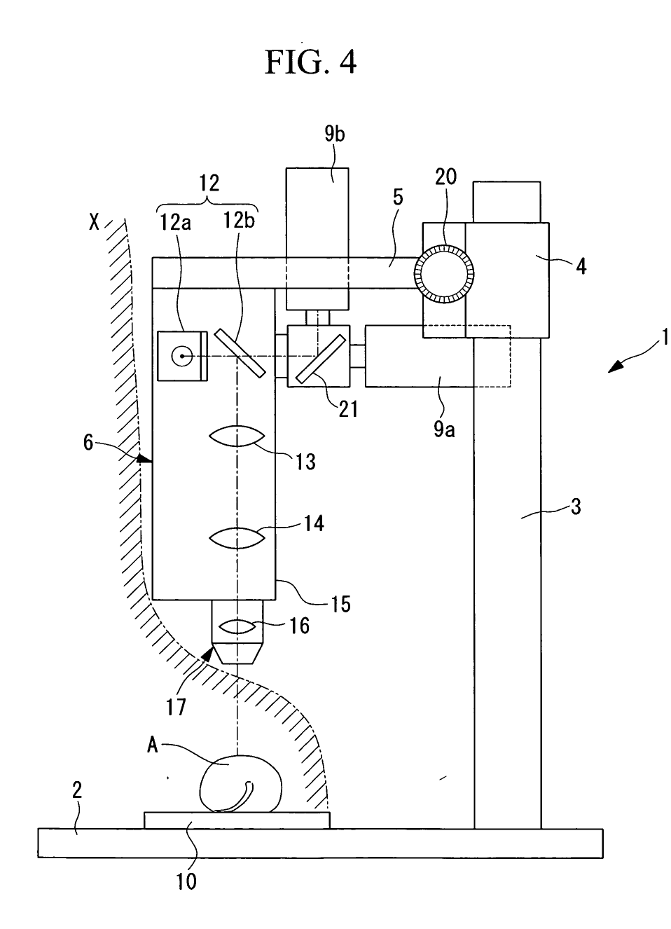

Moreover, with the laser-scanning examination apparatus 1 according to this embodiment, although a description has been given of a case where a single optical detector 9 is fixed to the surface at the support stand 3 side (the rear surface) of the casing 15 of the scan head 6, the invention is not limited to this configuration. For example, as shown in FIG. 4, a light splitter, such as a dichroic mirror 21, may be provided, and two or more optical detectors 9a and 9b that detect fluorescence of different wavelengths may be provided. In this case too, the working space X is only restricted by the optical detectors 9a and 9b at the rear side, but the working space X is not restricted at the front side, and thus examination can be carried out easily.

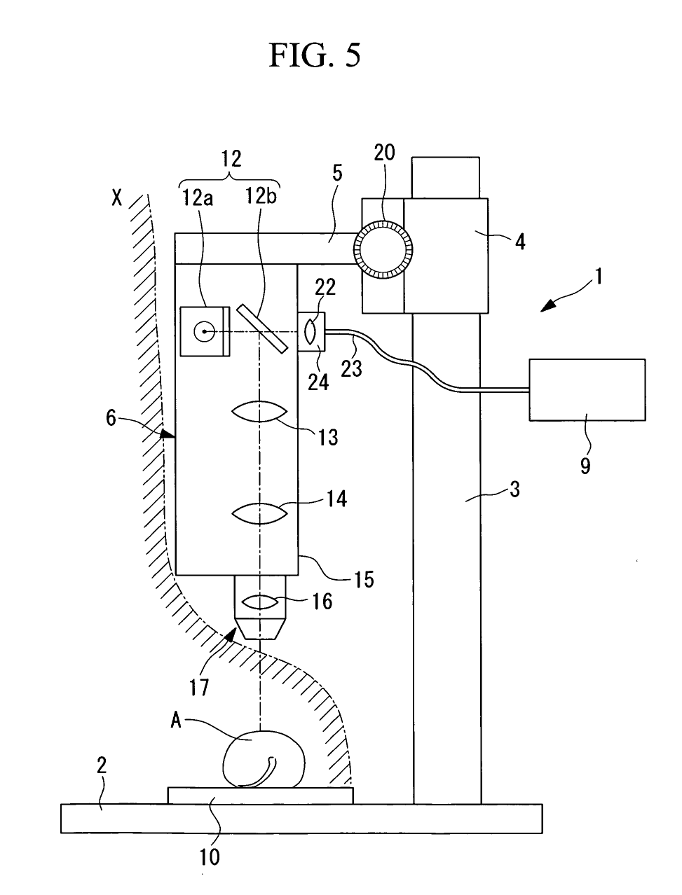

Furthermore, with the laser-scanning examination apparatus 1 according to this embodiment, a description has been given of the case where the optical detector 9 is directly fixed to the supports stand 3 side (the rear surface) of the casing 15 of the scan head 6. Instead of this configuration, however, as shown in FIG. 5, a separate optical detector 9 may be connected via a coupling lens 22 and an optical fiber 23. In this case too, it is possible to obtain the same advantages as described above by providing a connector 24, serving as a connecting part to the optical detector 9, in the rear surface.

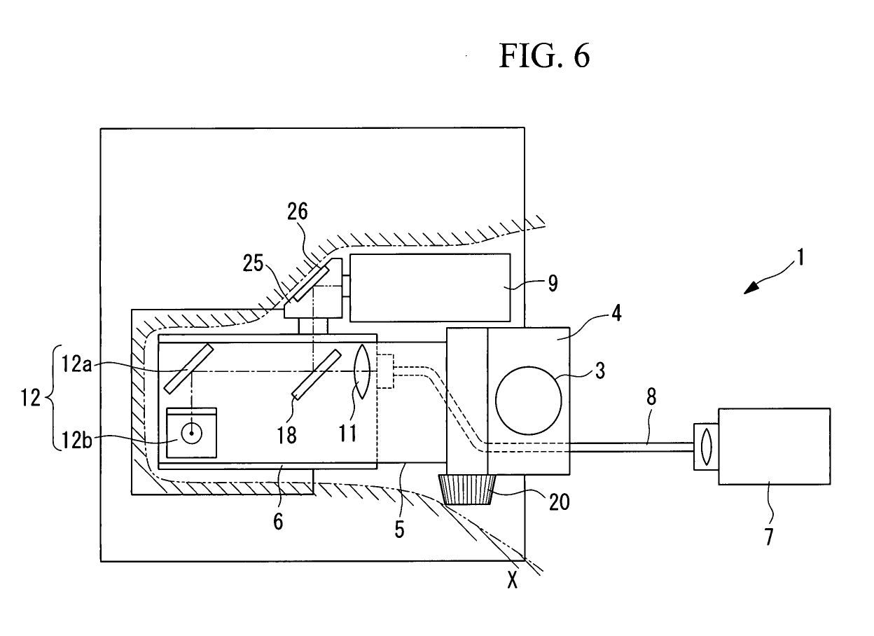

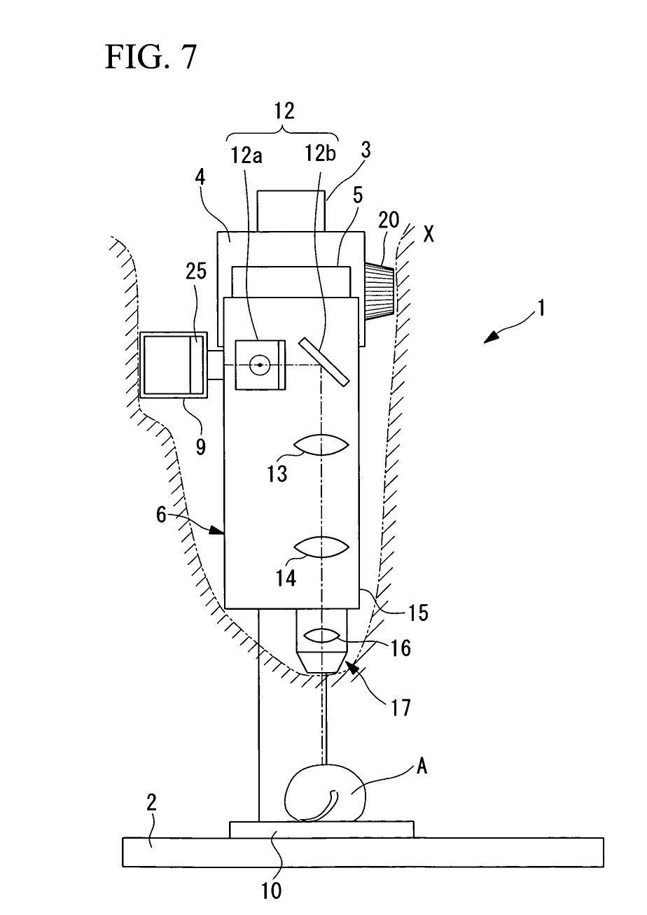

Moreover, in the embodiment described above, the casing 15 of the scan head 6 extends in the right and left directions when viewed from the front. However, instead of this configuration, as shown in FIGS. 6 to 8, if the casing 15 of the scan head 6 is aligned along the arm 5, the width dimension of the scan head 6, when viewed from the front, can be reduced. In this case, a connecting member 25 that guides the fluorescence split-off by the dichroic mirror 18 to the outside of the casing 15 is provided in one side surface at the left or right of the casing 15 so as to project therefrom. By reflection at a mirror 26 inside the connecting member 25, the fluorescence is again deflected in a direction parallel to the arm 5, and the optical detector 9 may be provided at the end of the support stand 3 side thereof so as to extend towards the support stand 3.

Since the connecting member 25 contains only the mirror 26, the amount by which it protrudes can be made sufficiently smaller than the optical detector 9, and therefore, it restricts the working space X at the left and right even less than the laser-scanning examination apparatus 1 shown in FIGS. 1 to 3. Also, since the optical fiber 8 connected to the laser light source 7 can be connected at the rear surface of the casing 15 in this case, the working space X can be further increased.

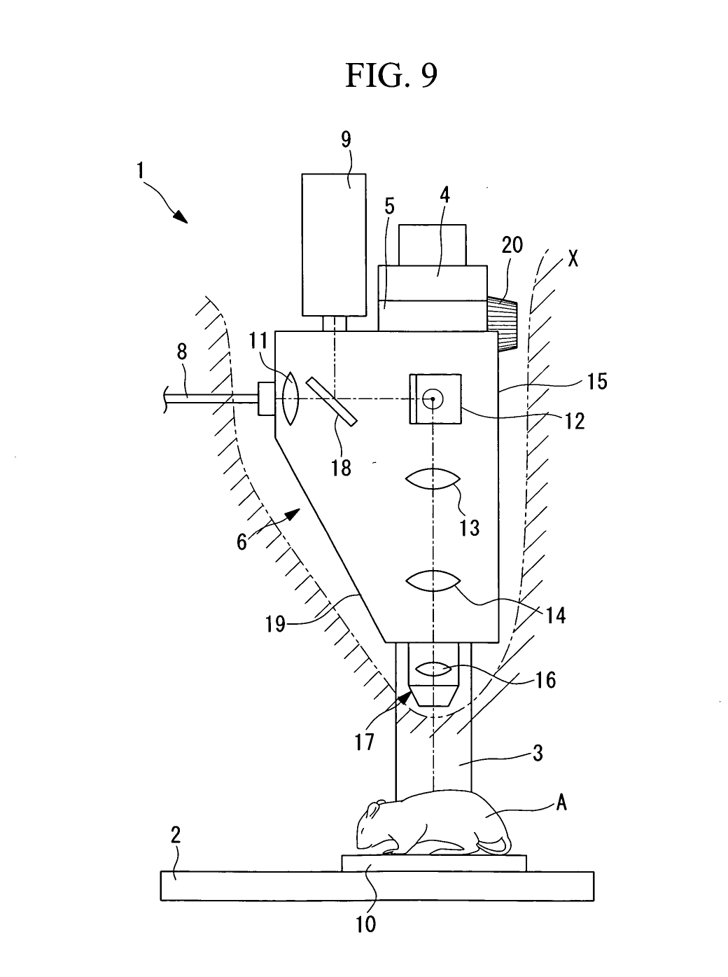

Also, in the embodiment described above, a description has been given of a case in which the optical detector 9 is provided at the rear surface of the casing 15 of the scan head 6; however, as shown in FIGS. 9 to 12, the optical detector 9 may be fixed to the upper surface of the casing 15.

FIG. 9, which is a view taken from the front side of the laser-scanning examination apparatus 1 according to this embodiment, like FIG. 2, shows the optical detector 9 connected to the upper surface of the casing 15 of the scan head 6, which projects towards the left from the arm 5.

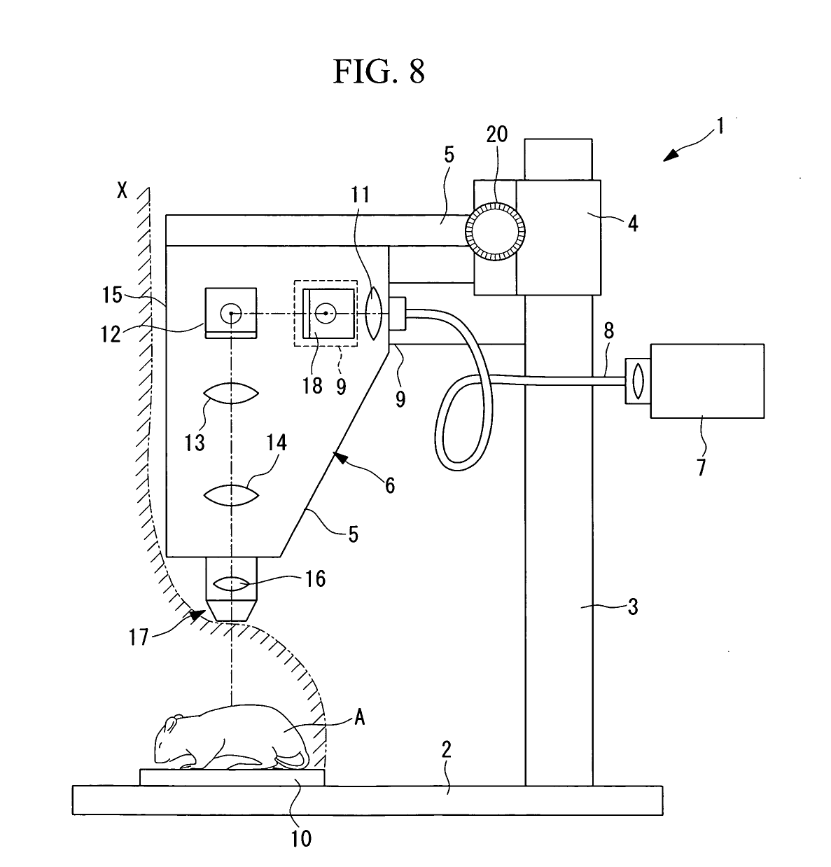

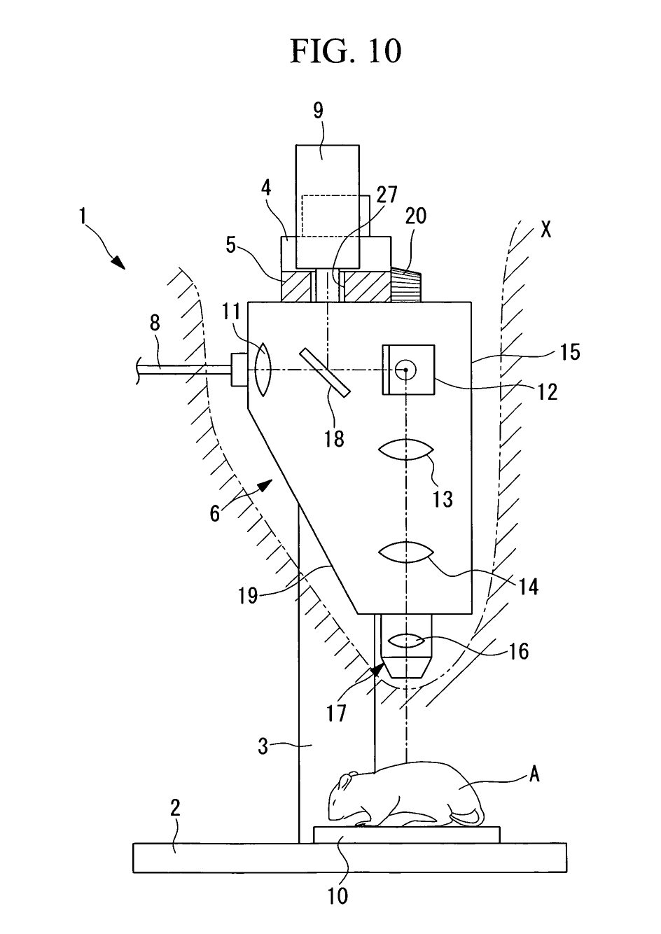

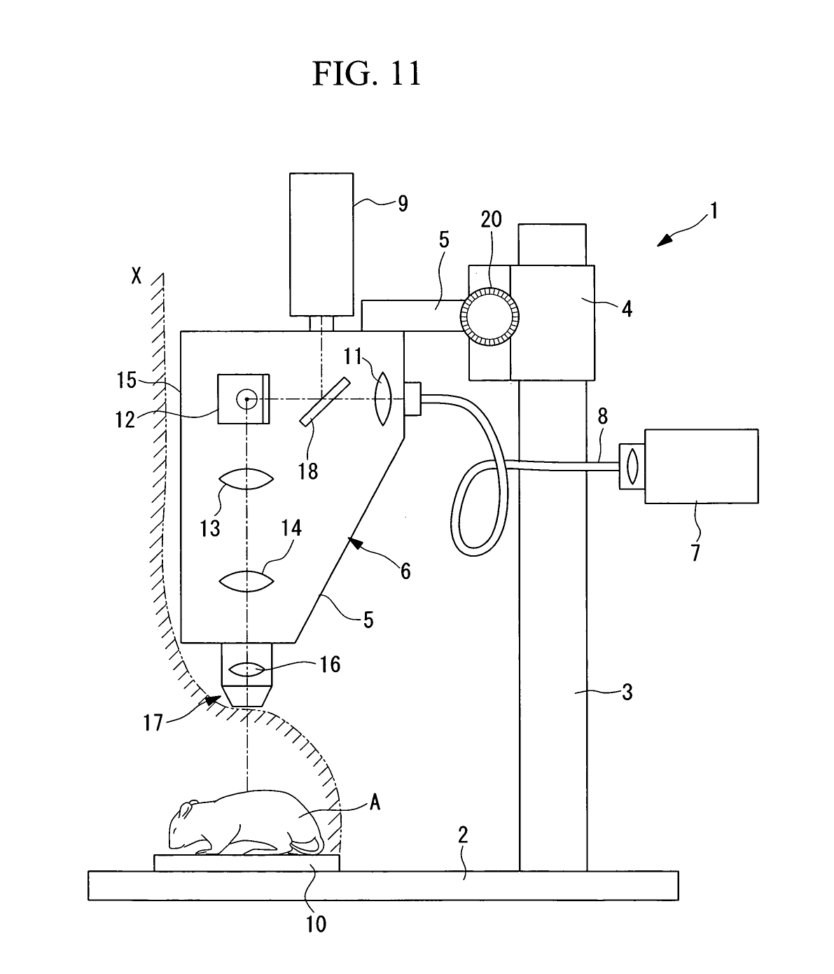

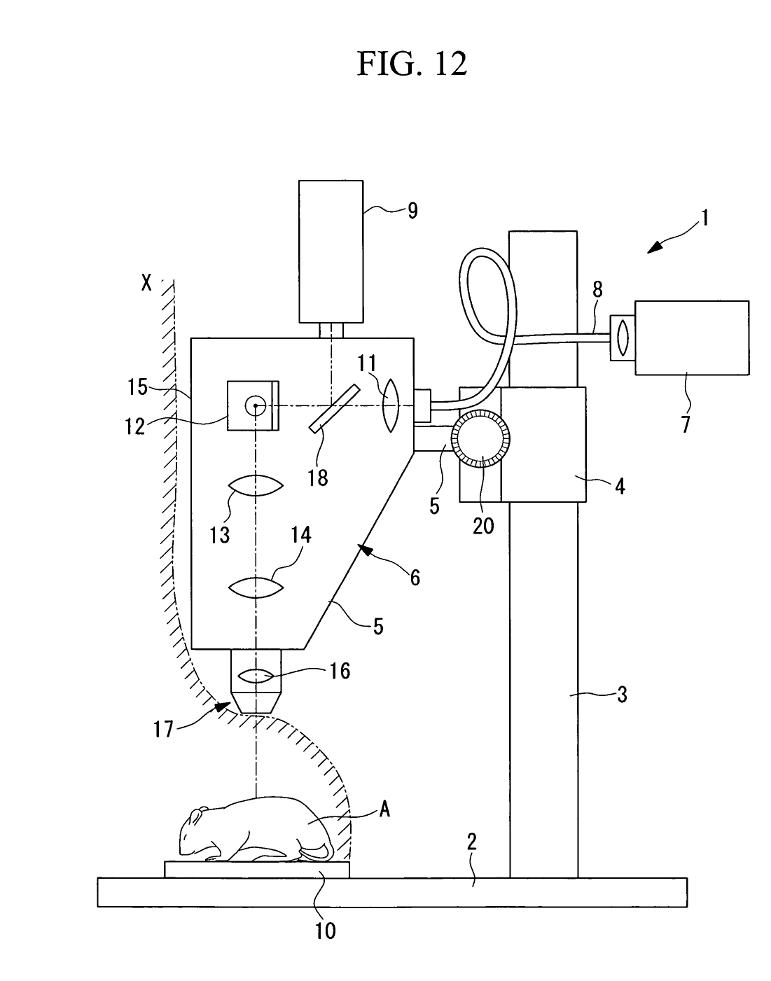

In FIG. 10, a through-hole 27 is provided in the arm 5, and the optical detector 9 is connected to the upper surface of the casing 15 of the scan head 6 via the through-hole 27. FIG. 11, which is a side view similar to that in FIG. 8, shows an example in which the casing 15 of the scan head 6 is fixed parallel to the arm 5 so as to reduce the width dimension when viewed from the front. The fixing region between the arm 5 and the casing 15 is made shorter to expose the upper surface of the casing 15 at the tip of the arm 5, and the optical detector 9 is fixed to the exposed upper surface of the casing 15. FIG. 12 is a side view, similar to FIG. 11, showing the fixing region between the arm 5 and the casing 15 disposed at the rear surface of the casing 15. With this arrangement, the upper surface of the casing 15 is completely exposed, and by fixing the optical detector 9 to this upper surface, the working space X is not restricted, in the same way as described above, and examination can thus be carried out easily.

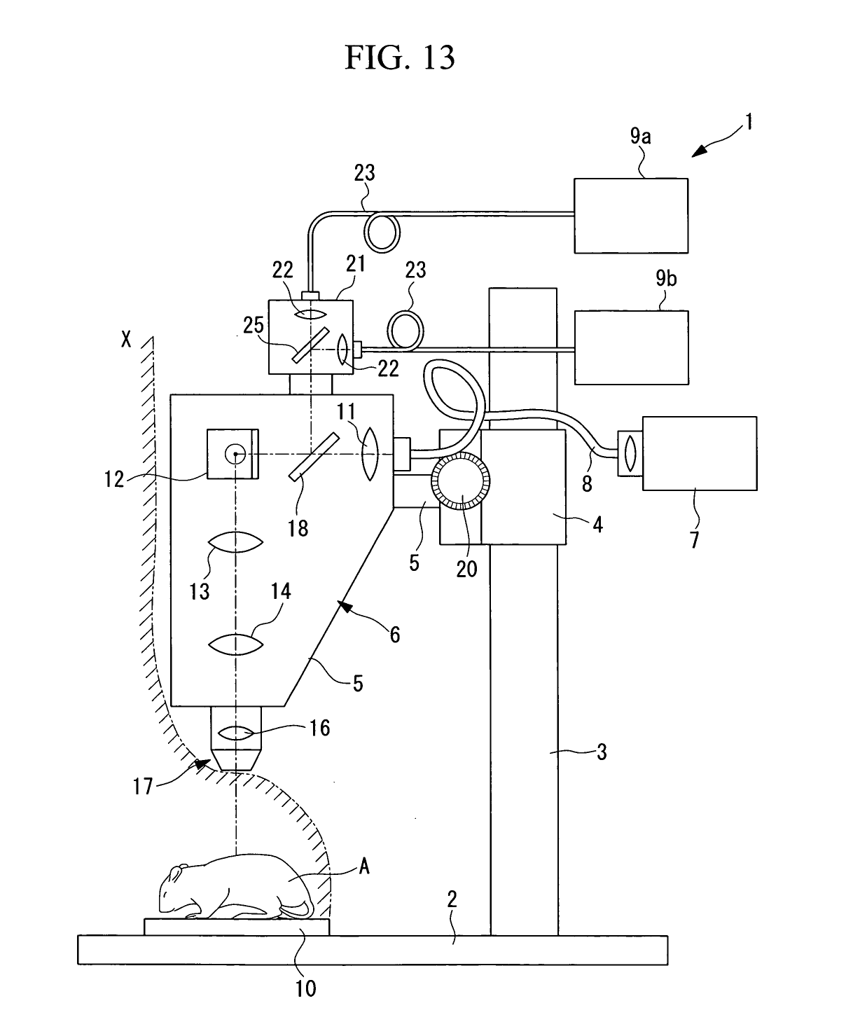

Moreover, apart from the case where the optical detector 9 is fixed directly to the upper surface of the casing 15, as shown in FIG. 13, optical detectors 9a and 9b may be connected via an optical splitter unit 21 and optical fibers 23.

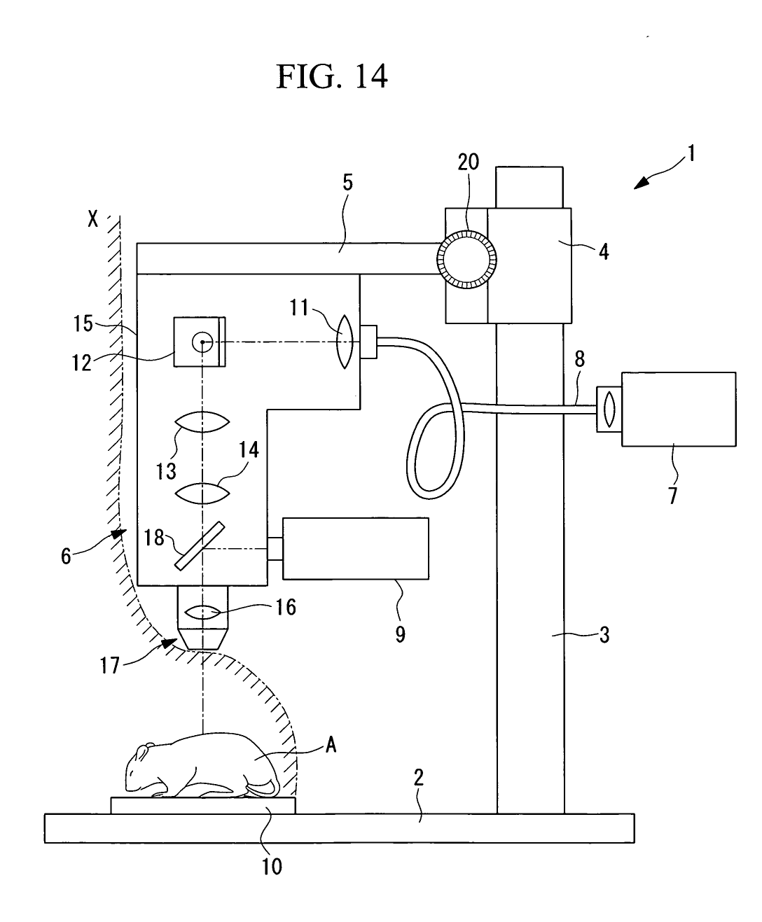

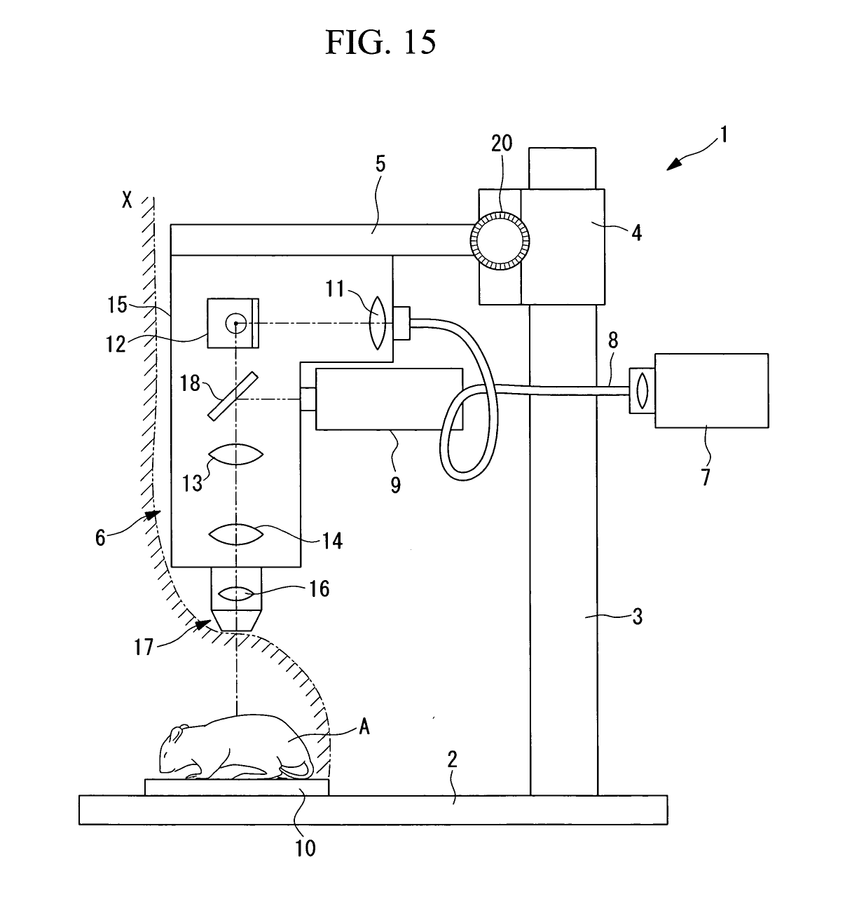

Furthermore, in the laser-scanning examination apparatus 1 according to the embodiment described above, the dichroic mirror 18 is disposed in the optical path between the laser scanning unit 12 and the collimator lens 11 and splits off the fluorescence returning from the specimen A. Instead of this configuration, however, as shown in FIGS. 14 and 15, the dichroic mirror 18 may be disposed in the optical path between the objective unit 17 and the imaging lens 14 or in the optical path between the pupil projection lens 13 and the laser scanning unit 12, to split off the fluorescence.

With these arrangements, the number of optical elements transmitting the fluorescence can be reduced. Accordingly, the fluorescence returning from the specimen A can be detected with reduced losses, thus suppressing deterioration of the image quality of the fluorescence image.

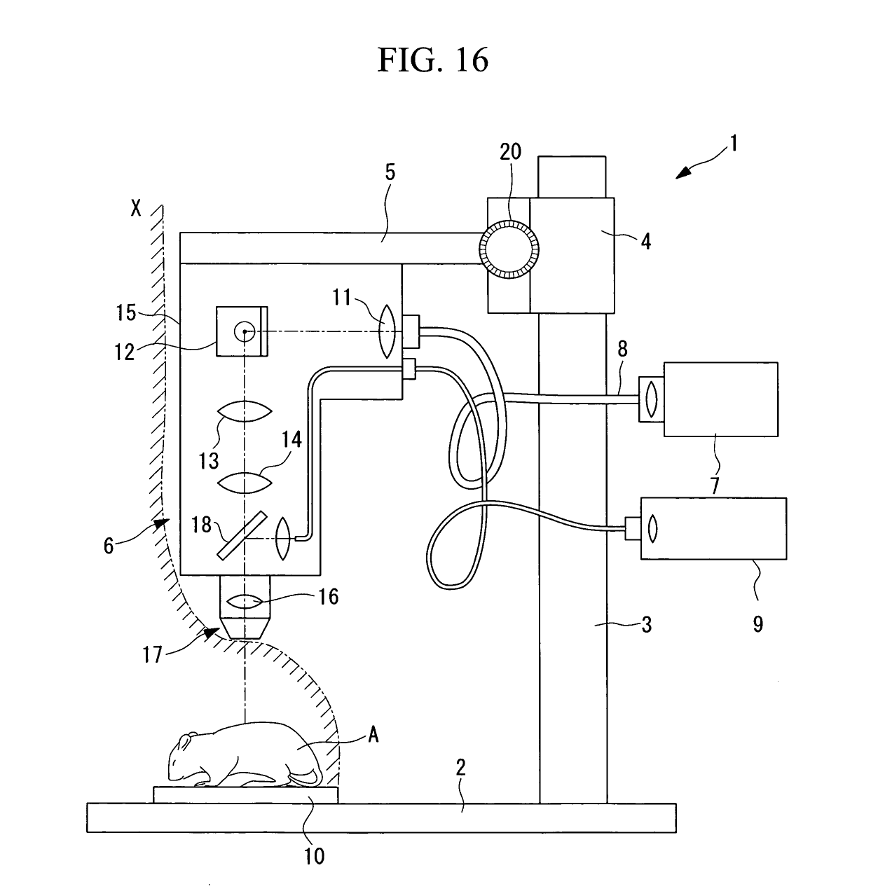

Furthermore, as shown in FIG. 16, in a case where the fluorescence is split off between the objective unit 17 and the imaging lens 14, a connector 24 serving as the connecting part between the optical detector 9 and the casing 15 can be disposed close to the location where the optical fiber 8, which conveys light from the laser light source 7, is connected to the casing 15. In this case, part of the optical fiber 8 from the connector 24 to the dichroic mirror 18 may be laid inside the casing 15 so that the tip of the optical fiber 8 faces the dichroic mirror 18.

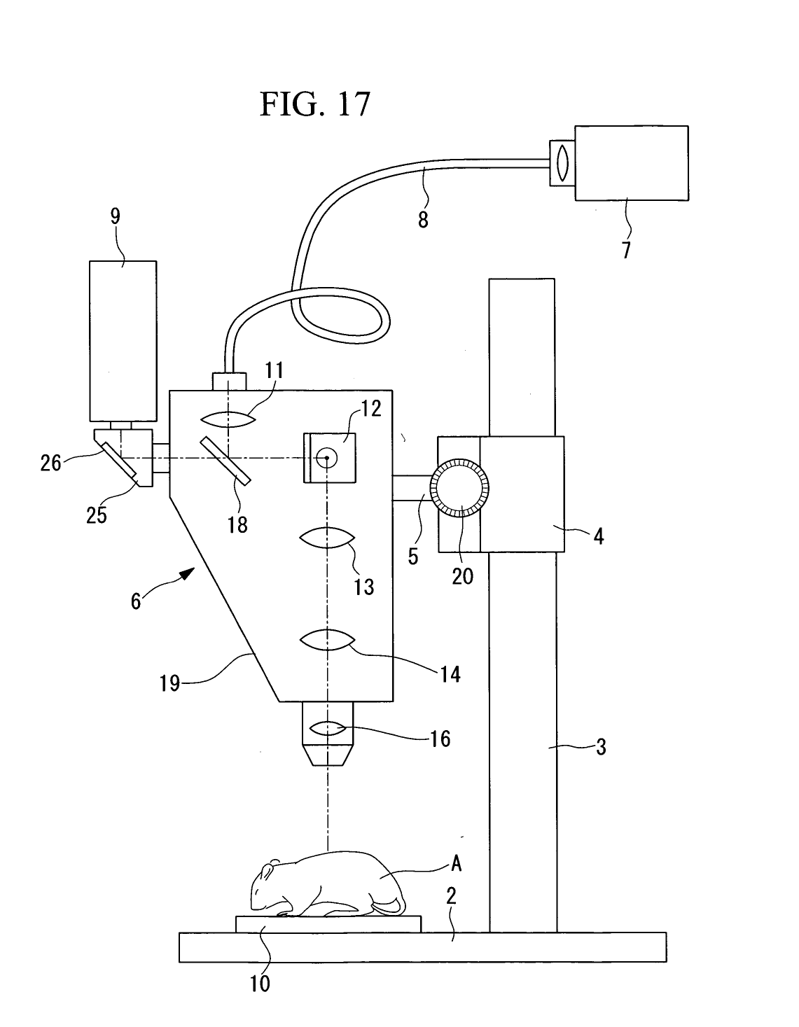

Moreover, as shown in FIG. 17, a connecting member 25 may be provided on the outer surface of the casing 15 disposed in the opposite direction to the direction in which the arm 5 extends from the casing 15, that is, in the surface disposed opposite to the support stand 3, and the optical detector 9 may be disposed higher than the upper surface of the casing 15. With this arrangement, the optical detector 9 can be placed at a position remote from the objective unit 17, which prevents working space X formed around the objective unit 17 from being reduced due to the optical detector 9.

When a confocal effect is to be obtained with the laser-scanning examination apparatus according to this embodiment, a method in which a pinhole for cutting defocus images is substituted at the core diameter of the optical fiber 8 and fluorescence is conveyed by the optical fiber 8 to be led to the optical detector 9 can also be considered. This method is useful mainly in the case of a single-photon-excitation examination apparatus, and the present invention is particularly effective in the case of a multi-photon-excitation examination apparatus in which a confocal effect is obtained without providing a pinhole for cutting defocus images.

### Second Embodiment

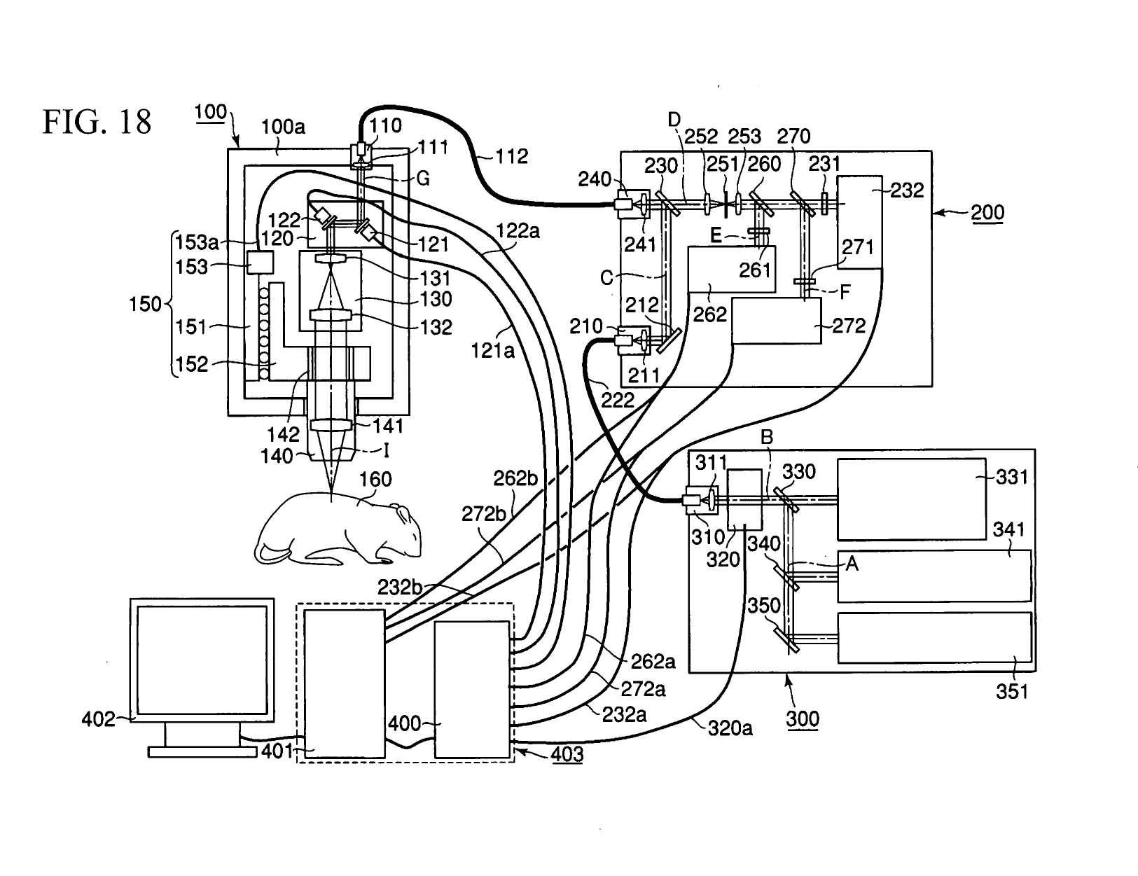

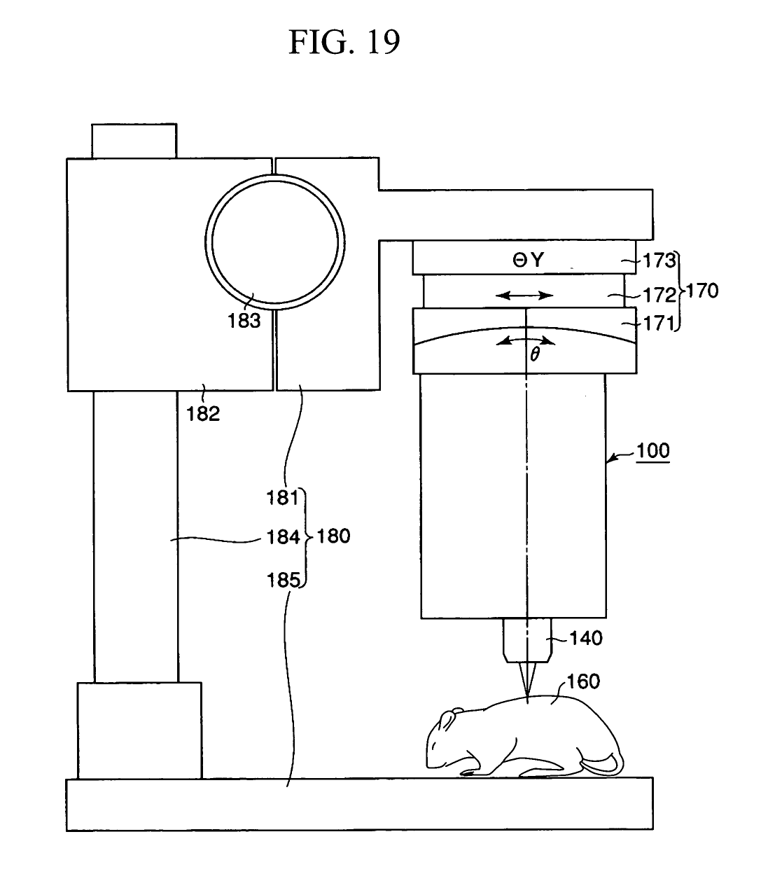

Next, a description of a laser-scanning examination apparatus according to a second embodiment of the present invention will be given below with reference to FIGS. 18 and 19.

FIG. 18 is a schematic diagram of a laser-scanning examination apparatus according to a second embodiment of the present invention.

In FIG. 18, reference numeral 100 represents a scan head, reference numeral 200 represents a detection apparatus, reference numeral 300 represents a laser generation apparatus, and reference numeral 403 represents a control unit.

The configuration of these individual components will be described in turn by following the path taken by the light along the optical axis.

The laser generation apparatus 300 is formed of laser light sources 331, 341, and 351 having different wavelengths, an AOTF (acousto-optic tunable filter) 320, dichroic mirrors 330 and 340, a reflecting mirror 350, and a connector 310 with a built-in lens 311.

The laser light emitted from the laser light source 341 is reflected onto the optical axis A shown in the figure by the dichroic mirror 340, which reflects this laser light and transmits the laser light from the laser light source 351. The laser light emitted by the laser light source 351 is reflected by the reflecting mirror 350 and transmitted by the dichroic mirror 340 to be combined on the optical axis A with the laser light from the laser light source 341. This combined laser light is then reflected at the dichroic mirror 330 onto the optical axis B shown in the figure, to be combined with the laser light emitted from the laser light source 331.

The laser light combined on the optical axis B and having different wavelengths is subjected to wavelength selection by the AOTF 320, and is then introduced into a second fiber 222 via the lens 311 in the connector 310. The AOTF 320 is electrically connected to a controller 400 via an AOTF cable 320a, so as to control the wavelength selection.

The detection apparatus 200 is connected to the second fiber 222. This detection apparatus 200 includes a connector 210. A collimator lens 211 is provided in this connector 210, for converting the diverging beam of light emitted from the second optical fiber 222 into a collimated beam. The collimated beam emitted from the collimator lens 211 is reflected at a reflecting mirror 212 onto the optical axis C shown in the figure, and is made incident on an excitation dichroic mirror 230. This excitation dichroic mirror 230 can be inserted in and removed from the detection apparatus 200. A component having a characteristic whereby the wavelengths of the laser light generated by laser light sources 331, 341, and 351 are reflected is selectively used as the excitation dichroic mirror 230.

The collimated light reflected at the excitation dichroic mirror 230 is reflected onto the optical axis D shown in the figure, is incident on a connector 240, and is introduced into a first fiber 112 via a coupling lens 241.

The scan head 100 is connected to the first fiber 112. This scan head 100 has a casing 100a and a connector 110 serving as a laser-light introducing part is provided so as to be fixed to the casing 100a. This connector 110 includes a collimator lens 111, which converts a diverging beam of light emitted from the first fiber 112 into a collimated beam.

The collimated beam emitted from the collimator lens 111 is introduced to a laser scanning unit 120 via an optical axis G shown in the drawing. The laser scanning unit 120 includes galvano mirrors (scanning mirrors) 121 and 122 which can be rotated around different rotation axes, and the collimated beam scanned by these galvano mirrors 121 and 122 is directed to an examination optical axis I shown in the figure. The galvano mirrors 121 and 122 are connected to the controller 400 via cables 121a and 122a, respectively, which allows their individual rotations to be controlled.

A second optical system 130 that is fixed in the casing 100a is disposed on the examination optical axis I. This second optical system 130 includes a pupil projection lens 131 and an imaging lens 132; after focusing the collimated beam directed onto the examination optical axis I with the pupil-projection lens 131, it is converted back to a collimated beam with the imaging lens 132.

The collimated beam from the second optical system 130 is directed to a first optical system 140. The first optical system 140 is supported in a detachable manner, by means of a securing thread 142, by a moving mechanism that is fixed to the casing 100a. Also, the first optical system 140 includes an objective lens 141, and light of a specific wavelength incident on the second optical system 130 is focused via this objective lens 141 onto a sample 160 as excitation light.

In this case, fluorescent proteins and so on that emit light (fluorescence) of specific wavelengths different from the excitation light by irradiation with the excitation light are introduced into the specimen 160. More concretely, the specimen 160 may be a mouse or rat in which a fluorescent protein or a fluorescent dye excited by near-infrared light is introduced on the surface or in the interior thereof, a human cancer cell in which a fluorescent protein is expressed, or an experimental animal such as a mouse or rat in which RNA is introduced.

The moving mechanism 150 includes a focus moving unit 152 that is moveable, parallel to the examination axis I, relative to a fixed unit 151 that is fixed to the casing 100a with screws or the like (not shown). By moving the focus moving unit 152 by means of a driving unit 153 provided at the fixed unit 151 side, the first optical system 140 can be moved along the examination axis I. The driving unit 153 is connected to the controller 400 via a focus cable 153a, which allows the amount of driving of the focus moving unit 152 to be controlled.

Regarding the positional relationships of the pupil projection lens 131, the imaging lens 132, and the objective lens 141, the pupil projection lens 131 and the imaging lens 132 are made coincident with substantially the central position of the galvano mirrors 121 and 122 and the back focal point of the objective lens 141, respectively. Also, the collimated beam from the galvano mirror 122 is arranged to be imaged at the position of the front focal point of the objective lens 141. With this arrangement, when the galvano mirrors 121 and 122 are rotated, the light beam incident on the pupil projection lens 131 is inclined, and as a result, the focal position of the objective lens 141 can be moved within a plane perpendicular to the examination optical axis I.

A casing moving mechanism 170 is connected to the upper surface of the casing 100a, that is, on a plane intersecting the optical axis including the objective lens 141. This casing moving mechanism 170 includes a tilting mechanism 171 for tilting the entire casing 100a in the direction of arrow 0 in the drawing, with the center of rotation being substantially the same position as the front focal position of the objective lens 141; and an X-axis moving mechanism 172 and a Y-axis moving mechanism 173 that respectively move the entire casing 100a in the X-axis and Y-axis directions in the drawing. Also, the casing moving mechanism 170 is supported by a microscope stand 180.

The microscope stand 180 includes a stand mounting part 182 mounted to a support stand 184 that is positioned upright on a base 185, and a focusing module 181 (arm) that can be moved, in a direction parallel to the support stand 184, relative to this stand mounting part 182 by means of a focusing knob 183. The casing moving mechanism 170 is supported by this focusing module 181.

The specimen 160 is held on a stage (not shown) of the base 185 of the microscope stand 180. How the specimen 160 is held is not shown, however.

The fluorescence emitted from the specimen passes back through the objective lens 141, the imaging lens 132, and the pupil projection lens 131, is reflected by the galvano mirrors 122 and 121, and is introduced into the first fiber 112 by the collimator lens 111. Fluorescence generated at parts other than where the light is focused on the specimen 160 cannot enter the first fiber 112.

The fluorescence passing through the first fiber 112 is incident on the collimator lens 240 in the detection apparatus 200, and passes through the coupling lens 241 to be converted to collimated light that propagates along the optical axis D. The collimated light then passes through the excitation dichroic mirror 230 and is incident on a focusing lens 252.

The focusing lens 252 focuses the collimated light and makes it incident on a pinhole 251. The pinhole 251 has an internal diameter ranging from one to three times the diameter of the light beam focused by the focusing lens 252. The fluorescence passing through the pinhole 251 is incident on a focusing lens 252 to be converted back to collimated light.

The pinhole 251 is adjusted in the direction of the optical axis D to substantially the same position as the focal position of the focusing lens 252, and so that its position in a plane orthogonal to the optical axis D is coaxial with the optical axis D.

A second dichroic mirror 260 and a third dichroic mirror 270 forming a fluorescence splitting unit are disposed on the light path of the fluorescence transmitted through the focusing lens 252. The second dichroic mirror 260 and the third dichroic mirror 270 can be inserted in and removed from the detection apparatus 200.

The second dichroic mirror 260 splits off light from the optical axis D to the optical axis E, and the third dichroic mirror 270 splits off light from the optical axis D to the optical axis F. A first optical detector 232 is disposed on the optical axis D of the light passing through the second dichroic mirror 260 and the third dichroic mirror 270, with a first absorption filter 231 positioned therebetween. A second optical detector 262 is disposed on the optical axis E of light split off by the second dichroic mirror 260, with a second absorption filter 261 positioned therebetween. A third optical detector 272 is disposed on the otical axis F of light split off by the third dichroic mirror 270, with a third absorption filter 271 positioned therebetween. The first absorption filter 231, the second absorption filter 261, and the third absorption filter 271 are designed to remove light of unnecessary wavelengths and transmit only fluorescence of specified wavelengths, thus introducing the fluorescence to the first optical detector 232, the second optical detector 262, and the third optical detector 272, respectively.

The first optical detector 232, the second optical detector 262, and the third optical detector 272 are connected to the controller 400 via a cable 232a, a cable 262a, and a cable 272a, respectively, so as to adjust the detection sensitivities thereof.

Also, the first optical detector 232, the second optical detector 262, and the third optical detector 272 are connected to detection ports (not shown) of a personal computer (hereinafter referred to as PC) 401 via a cable 232b, a cable 262b, and a cable 272b, respectively. Various types of software for controlling the controller 400 are installed on the PC 401, and this software can control each part, via the controller 400. Furthermore, the PC 401 processes fluorescence information from the first to third optical detectors 232, 262, and 272 to generate fluorescence images, which are then displayed on a monitor 402.

Next, the operating procedure of the second embodiment will be described.

First, in the software (not shown) installed in the PC 401, the operator sets the wavelength, intensity, examination region and so on of the laser light to be irradiated to the specimen 160. With these settings, the wavelength and transmission ratio of the light passing through the AOTF 320 and the rotation angle of the galvano mirrors 121 and 122 are set via the controller 400.

Next, when commencement of examination is selected using the software (not shown), the AOTF 320 is controlled, and a desired intensity of laser light from the laser light sources 331, 341, and 351 is guided along the optical path described above to be made incident on the specimen 160. At the same time, the galvano mirrors 121 and 122 start to rotate to scan the laser light (focus position) on the specimen 160 according to the examination region set in advance. In this state, when fluorescence is emitted from the specimen 160, the fluorescence from each examination position is guided to the first optical detector 232, the second optical detector 262, and the third optical detector 272, according to the wavelengths of the fluorescence, to be detected thereat. The corresponding fluorescence information is then transmitted to the detection ports (not shown) of the PC 401.

A fluorescence image is generated in the PC 401 based on the fluorescence information transmitted to the detection ports (not shown) and the scan position information of the galvano mirrors 121 and 122, and is displayed on the monitor 402 as a fluorescence image of the examination region set in advance. In this case, if the fluorescence image is dim or if the fluorescence intensity is too high, an appropriate fluorescence image can be obtained by adjusting the detection sensitivity of the first optical detector 232, the second optical detector 262, and the third optical detector 272 via the controller 400 with the software.

Next, setting of the examination position will be described.

In this case, the position in a plane orthogonal to the examination optical axis I is carried out by moving the entire casing 100a with respect to the specimen 160 by operating the X-axis moving mechanism 172 and the Y-axis moving mechanism 173; adjustment of the examination position parallel to the examination optical axis I is carried out by controlling the moving mechanism 150 with the software (not shown) via the controller 400 to move the entire first optical system 140 parallel to the examination optical axis I.

If the range of movement in the direction of the examination optical axis I is insufficient (if it cannot be adjusted with the moving mechanism 150 alone), the entire casing 100a, which is secured to the focusing module 181, can be moved relative to the specimen 160 by turning the focusing knob 183.

Furthermore, by moving the moving mechanism 150 by a small amount (10 nm to 1 μm) each time, after obtaining the fluorescence image as described above, a three-dimensional image can be displayed on the monitor 402 by superimposing multiple fluorescence images. Also, although it is necessary to incline the examination optical axis I depending on the examination position of the specimen 160, in this case, the entire casing 100a can be tilted using the tilting mechanism 171 to carry out examination.

Therefore, with this configuration, the examination position, angle, and so on of the scan head 100, provided, inside the casing 100a, with the connector 110 serving as the laser input section, the laser scanning unit 120 including the galvano mirrors 121 and 122, and the optical system including the objective lens 141, can be freely adjusted. Accordingly, this arrangement can relax the restrictions on the examination conditions, such as the examination orientation, of the specimen 160, and can provide a laser-scanning microscope that is best suited for examination of living organisms such as rats or mice.

Furthermore, in the scan head 100, since the first fiber 112 to which laser light from the laser light source is introduced is connected to a laser light input part (connector 110) securely disposed in the casing 100a, there is no unwanted movement of the first fiber 112 during scanning of the laser light, which prevents intensity variations in the laser light caused by movement of the fiber, thus allowing highly accurate examination images of experimental animals to be obtained.

Moreover, since the scan head 100 has a compact construction formed of the extremely small connector 110, the laser scanning unit 120 including the galvano mirrors 121 and 122, and the optical system including the objective lens 141, an apparatus that is small and easy-to-handle during examination can be realized.

In this second embodiment, three kinds of fluorescence can be simultaneously obtained using three laser light sources and three optical detectors. However, the same advantages as described above can be obtained even with a configuration including one laser light source and one optical detector. Furthermore, by irradiating the specimen 160 with laser light from a plurality of laser light sources simultaneously, it is possible to simultaneously and separately detect the fluorescence components with different wavelengths generated by the respective laser light wavelengths, and thus a multiple-wavelength-excitation, multiple-wavelength-detection technique such as FRET can be realized.

### First Modification of Second Embodiment

In the second embodiment described above, the AOTF 320 is used inside the laser generating apparatus 300; however, instead of this, a shutter (not shown) that can block the laser light and a light-intensity control device (not shown) that can attenuate the laser power may be provided in each laser light source, and these elements are controller by the controller 400.

In this case too, the same advantages as in the second embodiment can be obtained, and in addition, it is possible to provide a more inexpensive apparatus.

### Second Modification of Second Embodiment

In the second embodiment described above, the first fiber 112 and the second fiber 222 are separately prepared; however, it is possible to combine the first fiber 112 and the second fiber 222 into a multimode fiber.

With this configuration, in addition to providing the same advantages as in the second embodiment, adjustment of the fiber position is simplified, and there is a further advantage in that the light propagation efficiency is improved and the fluorescence detection sensitivity is enhanced.

Furthermore, the first fiber 112 and the second fiber 222 may be formed of crystal fibers.

### Third Modification of Second Embodiment

In the second embodiment described above, the first optical system 140 is secured to the moving mechanism 150 with the securing thread 142; however, the securing thread 142 may be an RMS thread and a microscope objective lens may be combined with the first optical system 140.

With this configuration, in addition to the same advantages as in the second embodiment, it is possible to combine objective lenses having various specifications, such as magnification, NA, and level of aberrations which improves the overall system performance.

### Fourth Modification of Second Embodiment

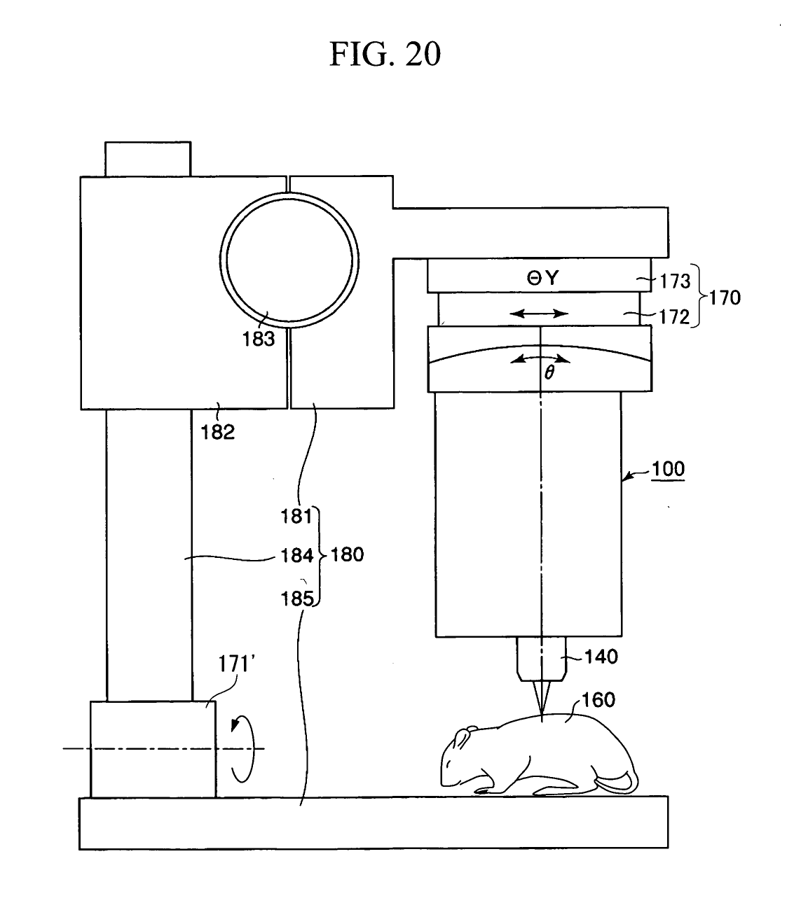

Moreover, in the second embodiment described above, the X-axis moving mechanism 172, the Y-axis moving mechanism 173, and the tilting mechanism 171 are disposed between the focusing module 181 and the scan head 100. Instead of this, however, as shown in FIG. 20, the X-axis moving mechanism 172 and the Y-axis moving mechanism 173 may be provided between the focusing module 181 and the scan head 100, and a tilting mechanism 171′ that pivots the support stand 184 around a horizontal axis with respect to the base 185 may be provided. Also, the apparatus may be configured so as to allow the tilting direction to be inclined in any direction by means of the tilting mechanisms 171 and 171′. Furthermore, the arrangement sequence of the tilting mechanism 171 and the X-axis and Y-axis moving mechanisms 172 and 173 may be set as desired.

### Third Embodiment

Next, a description of a third embodiment of the present invention will be given.

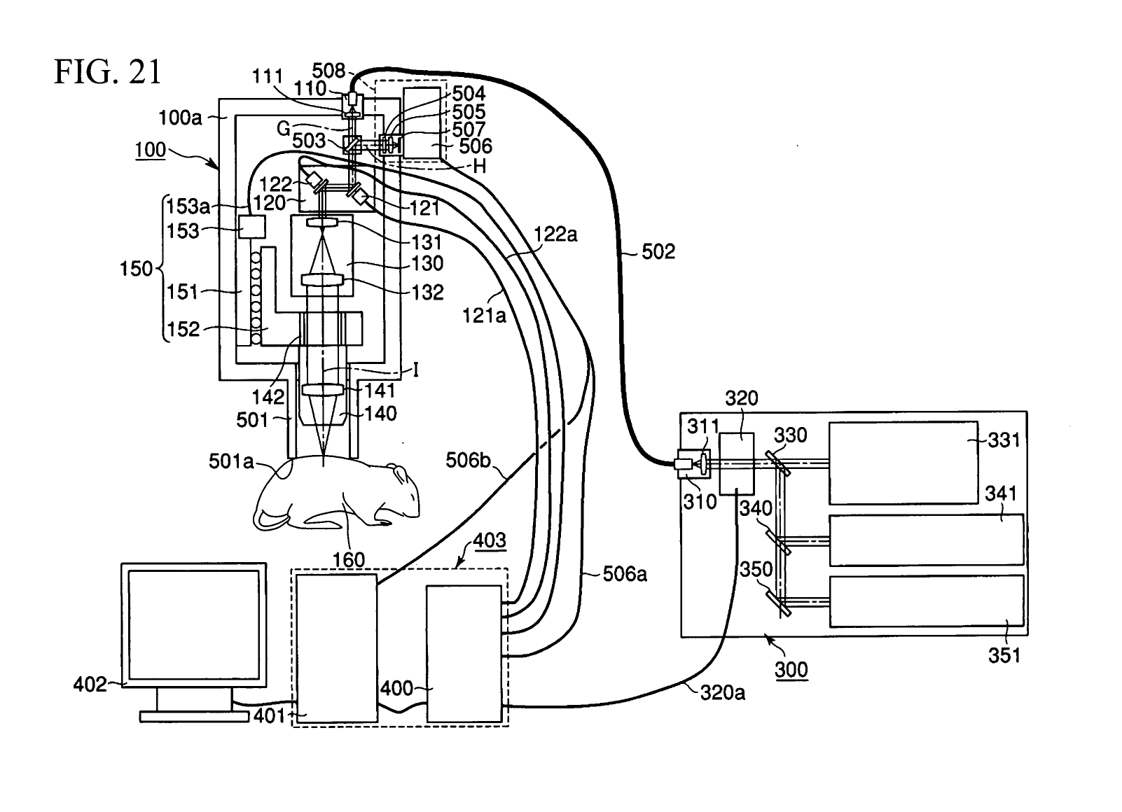

FIG. 21 is a schematic diagram of a laser-scanning microscope according to a third embodiment of the present invention, in which the same elements as shown in FIG. 18 are assigned the same reference numerals.

In FIG. 21, a scan head 100, a laser generating apparatus 300, and a control unit 403 are shown; however, since the basic functions of these elements are the same as those in the second embodiment, a description thereof is omitted.

In this case, a laser light of different wavelengths emitted from the laser generating apparatus 300 is introduced into a second fiber 502 via a lens 311 in a connector 310.

The scan head 100 is connected to the second fiber 502. A connector 110 is provided in a casing 100a thereof, and diverging light emitted from the second fiber 502 is converted to collimated light by a collimator lens 111 provided in this connector 110.

The collimated light emitted by the collimator lens 111 propagates along the optical axis G in the drawing and is incident on a laser scanning unit 120 that includes galvano mirrors 121 and 122. Then, the collimated light scanned with these galvano mirrors 121 and 122 is guided to the examination optical axis I shown in the drawing.

In this case too, the galvano mirrors 121 and 122 are connected to a controller 400 via cables 121a and 122a, which enables their respective rotations to be controlled.

The collimated light guided to the examination optical axis I is introduced to a second optical system 130 that is secured in the casing 100a; after being focused by a pupil projection lens 131, it is converted back to collimated light by an imaging lens 132.

Thereafter, the collimated light from the second optical system 130 is introduced to a first optical system 140. The first optical system 140 is supported in a detachable manner, by means of a securing thread 142, on a moving mechanism 150 secured to the casing 100a. Then, light of a specific wavelength is focused as excitation light onto a specimen 160, such as a mouse, via an objective lens 141 in the first optical system 140. In this case too, a fluorescent protein or the like that produces light (fluorescence) of a specific wavelength different from the excitation light is introduced into the specimen 160.

In this case, the casing 100a includes a protruding part 501 that protrudes in the direction of the examination optical axis I, and the first optical system 140 including the objective lens 141 is located in a hollow portion inside this protruding part 501. The protruding part 501 is arranged such that a tip 501a thereof is placed in contact with the surface of the specimen 160, and in this state, the excitation light is irradiated onto the specimen 160 via the objective lens 141.

The fluorescence emitted from the specimen 160 passes back through the objective lens 141, the imaging lens 132, and the pupil projection lens 131, and is reflected by the galvano mirrors 122 and 121 to be guided onto the optical axis G.

An insertable/removable dichroic mirror 503 is disposed between the collimator lens 111 and the laser scanning unit 120. The dichroic mirror 503 has a characteristic whereby it transmits laser light emitted from the collimator lens 111 and reflects the fluorescence emitted from the specimen 160.

With this configuration, the fluorescence reflected by the galvano mirrors 122 and 121 is reflected by the dichroic mirror 503 to be directed onto the optical axis H shown in the drawing.

An optical detection unit 508 that can be attached to and removed from the casing 100a is disposed on the optical axis H. The optical detection unit 508 includes an absorption filter 504, a focusing lens 505, a pinhole 507, and an optical detector. The fluorescence directed onto the optical axis H is filtered by the absorption filter 504 to remove unwanted light and is introduced to the focusing lens 505. Unwanted light is removed from the light focused by the focusing lens 505 by means of the pinhole 507, which has an inner diameter from one to three times the beam diameter, and the light is then introduced to the optical detector 506. The pinhole 507 is adjusted in the direction of the optical axis H to substantially the same position as the focal position of the focusing lens 505, and so that its position in a plane orthogonal to the optical axis H is coaxial with the optical axis H.

The optical detector 506 is connected to the controller 400 via a cable 506a, and is connected to a detection port (not shown) of a PC 401 via a cable 506b.

In this case too, various types of software for controlling the controller 400 are installed on the PC 401, and this software can control each component, via the controller 400. Furthermore, the PC 401 processes fluorescence information from the optical detector 506 to generate a fluorescence image, which is then displayed on the monitor 402.

Next, the operation of the third embodiment will be described.

To carry out examination in this case, first, the position of the casing 100a is adjusted so as to bring the tip 501a of the protruding part 501 of the casing 100a into contact with the specimen 160. The method of carrying out this position adjustment is the same as that described in the second embodiment.

Next, similar to the second embodiment, the wavelength, intensity, examination region and so on of the laser light are set using software (not shown). Thereafter, an instruction to commence examination is given, and adjustment of the detection sensitivity of the optical detector 506, adjustment of the examination position, and so forth are carried out. By doing so, when fluorescence information is transmitted from the optical detector 506 to a detection port (not shown) of the PC 401, a fluorescence image is generated, using scan-position information of the galvano mirrors 121 and 122, and this image is displayed on the monitor 402 as a fluorescence image for the examination region that is specified in advance.

Accordingly, with a compact configuration that is suitable for examination of a living body such as a mouse or rat, it is possible to provide a laser-scanning microscope that can freely adjust the examination position and angle. In this case, since the fluorescence from the specimen 160 can be introduced to the optical detector 506 without going via a fiber, it is possible to reliably detect even weak fluorescence.

Also, since the tube-shaped protruding part 501, which protrudes in the direction of the examination optical axis I of the casing 100a, is pressed against the specimen 160, it is possible to perform examination in a stable state in which the specimen 160 is secured so as not to move. Moreover, since the objective lens 141 is disposed in the hollow portion inside the tube-shaped protruding part 501, it is possible to prevent deterioration of the fluorescence image caused by unwanted light (room light etc.) getting into the objective lens 141.

Furthermore, since the moving mechanism 150 is disposed inside the casing 100a and it is possible to move the first optical system 140 in the direction of the examination optical axis I inside the tube-shaped protruding part 501, it is possible to keep the tip 501a of the tube-shaped protruding part 501 pressed against the specimen 160 even when adjusting the examination position in the direction of the examination optical axis I. Accordingly, when performing in-vivo examination of a mouse or the like, it is possible to prevent application of an unnecessary load to the specimen 160.

In this third embodiment, an example is shown in which three laser light sources are combined; however, the same advantages as described above can be obtained by configuring the apparatus with a single laser light source.

### Modification of Third Embodiment

In the third embodiment described above, the dichroic mirror 503 and the optical detector unit 508 including the absorption filter 504, the focusing lens 505, the pinhole 507, and the optical detector 506 are disposed on the optical axis H. However, at least one optical detector unit exactly the same as this can be used, disposed on the optical axis G or the optical axis H. In this case, the detection sensitivity of the optical detector of the additional optical detector unit can be controlled from the controller 400, like the optical detector unit 508, and the detected fluorescence information is output to the PC 401 via a cable 506b.

With this configuration, the specimen is irradiated with laser light having different wavelengths, and fluorescence of different wavelengths corresponding to the respective irradiation wavelengths is separated and detected with the optical detector unit 508. A multi-wavelength excitation, multi-wavelength detection technique such as FRET can thus be realized.

The present invention is not intended to be limited to the embodiments described above. In practicing the invention, various modifications within a scope that does not depart from the substance thereof are possible.

Furthermore, the embodiments described above include various aspects of the invention, and various aspects of the invention can be obtained by suitably combining the plurality of disclosed structural elements. For example, even when various structural elements are removed from the complete structure disclosed in the embodiments, so long as the problems described above in the Summary of the Invention can be overcome and the advantages described therein can be obtained, the configuration from which these structural elements are removed can be considered as the invention.

1. A laser-scanning examination apparatus comprising:
a laser light source;
an irradiation optical fiber through which laser light generated in the laser light source is transmitted;
a scan head including a laser scanning unit for scanning the laser light transmitted through the irradiation optical fiber on a specimen, a casing for housing the laser scanning unit, and an objective unit for imaging the laser light scanned by the laser scanning unit onto the specimen;
an optical detector for detecting returning light that returns from the specimen to the interior of the casing via the objective unit;
a stage for mounting the specimen;
a base on which the stage is provided;
a support stand having a longitudinal axis extending from the base;
an arm that extends from the support stand in a direction orthogonal to the longitudinal axis; and
a moving mechanism disposed between the arm and the scan head,
wherein the moving mechanism moves the scan head relative to the arm.

2. The laser-scanning examination apparatus according to claim 1, wherein the scan head is attached to the moving mechanism at an outer surface of the casing orthogonal to an optical axis of the objective unit.

3. The laser-scanning examination apparatus according to claim 1, comprising a support stand tilting mechanism for tilting the support stand about a horizontal axis thereof.

4. The laser-scanning examination apparatus according to claim 1, wherein the moving mechanism includes an X-axis and Y-axis moving mechanism for moving the scan head relative to the arm in two directions orthogonal to the optical axis of the objective unit.

5. A laser-scanning examination apparatus according to claim 1, wherein the moving mechanism includes a tilting mechanism for adjusting the tilt angle of the scan head with respect to the arm.

6. A laser-scanning examination apparatus according to claim 1, comprising a focus adjusting mechanism for relatively moving the objective unit along the optical axis thereof with respect to the casing.

7. The laser-scanning examination apparatus according to claim 1, wherein the optical detector is provided on an outer surface of the casing except for the surface positioned in a direction opposite to the direction in which the arm extends from the casing.

8. The laser-scanning examination apparatus according to claim 7, wherein the optical detector is secured to the casing in a direction extending along the arm.

9. The laser-scanning examination apparatus according to claim 7, comprising:
a slider that is moveable upwards and downwards along the support stand;
wherein the base is disposed horizontally, the support stand extends vertically from the base, and the arm is attached to the slider; and
wherein the optical detector is attached to the outer surface of the casing so as to extend in the direction of the support stand.

10. The laser-scanning examination apparatus according to claim 7, wherein the casing is provided with an inclined surface formed so as to taper towards the objective unit.

11. The laser-scanning examination apparatus according to claim 7, wherein the optical detector is disposed at the opposite side of the objective unit from the stage.

12. The laser-scanning examination apparatus according to claim 1, wherein the optical detector is positioned higher than the top surface of the casing.

13. The laser-scanning examination apparatus according to claim 1, comprising:
a connecting part for connecting to the optical detector, the connecting part being provided on an outer surface of the casing except for the surface located in the opposite direction from the direction in which the arm extends from the casing.

14. The laser-scanning examination apparatus according to claim 13, comprising:
a light-path splitting unit, in the connecting part, for splitting the light path;
wherein the optical detector includes a plurality of detectors for detecting light in the respective split light paths.

15. The laser-scanning examination apparatus according to claim 13, wherein the connecting part is disposed towards the support stand side of the optical axis of the objective unit.

16. The laser-scanning examination apparatus according to claim 13, comprising:
a detection optical fiber that connects the connecting part and the optical detector.

17. The laser-scanning examination apparatus according to claim 1, comprising:
a splitting unit for splitting off returning light from the optical path between the objective unit and the laser scanning unit and for directing the split-off light towards the optical detector.

18. The laser-scanning examination apparatus according to claim 13, comprising:
a splitting unit for splitting off returning light from the optical path between the objective unit and the laser scanning unit and for directing the split-off light towards the optical detector; and
a detection optical fiber that connects the connecting part and the optical detector,
wherein the connecting part is disposed near the position where the irradiation optical fiber is connected to the casing, and
wherein part of the detection optical fiber is disposed inside the casing so that one end thereof opposes the splitting unit.
